(19) United States
(12) Patent Application Publication
(10) Pub. No.: US 2007/0292402 A1
(43) Pub. Date: Dec. 20, 2007

(54) Lyophilised Lactic Acid Bacteria And Bifidobacteria Products With A High Degree Of Dispersibility

(76) Inventors: Franco Pirovano, Sulbiate (IT); Giorgio Reiner, Como (IT)

Correspondence Address: LUCAS & MERCANTI, LLP, 475 PARK AVENUE SOUTH, 15TH FLOOR, NEW YORK, NY, 10016, US

(21) Appl. No.: 11/576,021

(22) PCT Filed: Sep. 28, 2005
(86) PCT No.: PCT/EP05/54859

§ 371 Date: Jun. 20, 2007

(30) Foreign Application Priority Data
Oct. 15, 2004 (IT) RM2004A000505

(51) Int. Cl. A61K 35/12; A23C 23/00; A61P 1/00; C12N 1/20; A23L 1/00

(52) U.S. Cl. 424/93.45; 426/61; 435/252.9; 435/253.4

(57) ABSTRACT

Lyophilised lactic acid bacteria and bifidobacteria products are described, characterised by a high degree of dispersibility thanks to the use of polyalcohols and/or sugars as dispersing agents. The liquid dispersions of these lyophilised products are characterised by a high degree of stability.

The present invention relates to lyophilised products containing lactic acid bacteria and bifidobacteria used in the food field, and more particularly in the nutritional and pharmaceutical fields. Thanks to the present invention, which improves the technical aspect of the wettability of lactic acid bacteria and bifidobacteria, the products containing them are characterised by improved stability and drinkability characteristics.

### BACKGROUND TO THE INVENTION

Lactic acid bacteria, otherwise called lactobacilli or lactobacteria, and bifidobacteria are extensively used in the food, nutritional and pharmaceutical fields, thanks to their multiple beneficial activities.

One of the most interesting aspects of lactic acid bacteria and bifidobacteria is their use as ‘starters’, whether lyophilised or not, in the production of various dairy products. Given their poor or non-existent solubility, there is the problem of their dispersion in aqueous media, which, in this field, presents itself in the form of having to disperse a small amount of lyophilised product in a large amount of liquid product (e.g. milk) to be transformed.

One of the first modes of administration of lactobacilli and bifidobacteria, in addition to the ‘natural’ one via the consumption of yogurt, consists in suspending them in a drinkable liquid medium. This suspension has particular organoleptic characteristics and a generally milky appearance, rendering it poorly acceptable to children and adults who tend to associate it with a drug, thus reducing its consumption to cases of strict necessity and at the same time causing them to lose the prophylactic and preventive effect of probiotics.

Studies of the beneficial characteristics of lactic acid bacteria and bifidobacteria and the demonstration of their therapeutic efficacy in a series of diseases have prompted research into the most acceptable administration forms from the organoleptic point of view, which at the same time are endowed with greater lasting stability. In fact, lactic acid bacteria and bifidobacteria need to be viable in order to exert their efficacy, which makes it mandatory to adopt certain conservation pre-cautions on the part of the distribution chain and the end user.

In particular, in the nutritional or dietary supplement field, and more particularly in the so-called “nutraceuticals” field or in that of actual drugs, where the purpose of the prevention and cure of frankly diseased states is much more pronounced, the tendency, and indeed we would say the need, is to provide very high concentrations of lactobacilli per dosage unit. Typically, the bottom limit for lactobacilli concentrations is 10⁹ CFU/g.

Examples of the use of high-concentration lactic acid bacteria and bifidobacteria, also in the form of lyophilised products, are to be found in the litereature. See, for example, EP 0 555 618, U.S. Pat. No. 6,258,355, U.S. Pat. No. 6,326,000, U.S. Pat. No. 6,582,695, WO 03/055984, and U.S. Pat. No. 6,572,854.

Lyophilisation is an elective technique for the conservation of lactobacilli, using particular process conditions which do not impair the viability of the micro-organisms.

The phases during lyophilisation are strictly conditioned by the nature and state of the material to be subjected to lyophilisation, as well as by the equipment used.

At the end of the process, the product thus treated maintains its chemical, physical and biological properties.

In the lyophilisation of bacteria (for food, dietetic, probiotic or therapeutic purposes), one of the aspects that always needs to be carefully assessed is the choice of substance, or mixture of substances, to be added to the cellular biomass for the protection (reduction of the mortality) of cells during the various lyophilisation phases, particularly the freezing phase, and for improving the duration of conservation.

Various substances can normally be used for protection, such as;
polysaccharides (e.g. dextrans);
monosaccharides (e.g. fructose, glucose);
disaccharides (e.g. lactose, maltose, sucrose);
trisaccharides (e.g. raffinose);
alcohols and polyalcohols (e.g. inositol, sorbitol, glycerol);
proteines (e.g. casein, ovalbumin);
others (e.g. monosodium glutamate, ascorbic acid);
buffer substances.

All these substances can be used either singly or in mixtures with different proportions.

Essentially, they are used to protect the bacterial cell during the freezing phase and thus to reduce cell mortality and create an adequate physical structure of the product subsequently favouring the sublimation of the water during the lyophilisation phase.

Lyophilisation techniques are now currently used in the manufacture of probiotic products based on lactic acid bacteria and bifidobacteria, but their delicacy in terms of stability presupposes the need to choose the best lyophilisation support.

Usually, these products are formulated in the form of powders, which are taken after being dissolved in a liquid.

Lyophilised lactic acid bacteria and bifidobacteria products are characterised by very low solubility and the excipients normally used to stabilise the bacterial load can also make the situation worse. Among these excipients, we can mention lactose, starch and maltodextrines.

Given their generally disagreeable taste, these lyophilised products are dissolved in a minimum amount of liquid and drunk. The extemporaneous formulation of a suspension with a high concentration of lactic acid bacteria does not appear very attractive to those taking the product.

Once dispersed in a small amount of water, the product tends to form agglomerates or clumps, being wetted only on the surface. These agglomerates, also according to their specific weights, may remain on the surface of the liquid, or may lie on the bottom of the container or even become attached to its walls. Its consumption thus proves difficult and often the residue has to be re-amalgamated with water in the container, mixed again and drunk a second time in order to take the complete dose, experiencing once again the unpleasant taste.

This difficult formulation of the lyophilised product poses several problems with regard to its consumption by children, who do not always accept the disagreeable taste, or elderly subjects who cannot drink quickly or who present difficulty with the intake of large amounts of liquids in general.

Formulating the lyophilised probiotoic product in larger amounts of liquid, while, on the one hand, attenuating the problem of precipitation and adhesion to the walls of the container, on the other worsens the organoleptic sensation by prolonging it. Formulation in other media, e.g. fruit juices, milk, or some other beverage, may improve the palatability of the product, but does not solve the problem of its wettability; indeed, in some cases, this may even be aggravated by the presence of certain substances in the medium.

The problem facing the expert in the field is that of providing a lactobacilli- or bifidobacteria-based probiotic product which is endowed with acceptable dispersion in a small amount of liquid.

This problem, which is present both in the field of probiotic products and in the strictly alimentary field, such as, for example, in the manufacturing process of cheeses, yogurts, fermented milks, oven products, meat products, and vegetable products, where either fermentation or the addition of bacteria is required, has yet to be satisfactorily solved, but above all it has not been entirely solved for lactic acid bacteria and bifidobacteria concentrations greater than 10⁹ CFU/g, this already being a concentration at which, to the best of the inventors' knowledge, dispersion of the bacteria is not optimal.

The same problem is encountered in the manufacture of cheeses, mozzarella cheese, yogurt, etc.

### SUMMARY OF THE INVENTION

It has now surprisingly been found that the use of polyalcohols and/or sugars in lactobacilli- and bifidobacteria-based lyophilised products in which a bacterial load of 10⁹ CFU/g is present, gives rise to a product which is rapidly dispersed in the medium thanks to its increased wettability; this reduces the large agglomerates wetted only superficially and, as explained above, capable of causing the disagreeable phenomenon of attachment to the walls of the container or even precipitation. Moreover, the residual precipitation proves to be considerably reduced and, in any event, easily re-dispersable.

Therefore, one subject of the present invention is a lyophilised product containing lactobacilli and bifidobacteria in a concentration equal to or greater than 10⁹ CFU/g, characterised in that it contains one or more polyalcohols and/or sugars as dispersion agents.

This leads to an immediate advantage in the formulation of dairy products, in which a small amount of lactobacilli needs to be homogeneously dispersed in a very large volume.

As regards the products in the nutritional and pharmaceutical fields, the present invention provides a product which is easy to take and does not require any particular mixing, or re-amalgamation to complete its consumption, or at least requires it to a much lesser, if not entirely negligible extent.

The lactobacilli- or bifidobacteria-based probiotic product, lyophilised in the presence of dispersing agents according to the present invention, is endowed with several advantages over any similar products known to date. As mentioned earlier, the lyophilised product is dispersed very rapidly in the drinking liquid, with only a minimal stirring action so that most of the product is consumed in the first few mouthfuls, unlike what happens with the known products. Another advantage is the fact that once the lyophilised product has been drunk, only a negligible amount of it remains attached to the walls of the container, with the result that it is not necessary to re-amalgamate it with more liquid.

### DETAILED DESCRIPTION OF THE INVENTION

The lyophilised probiotic product according to the present invention is obtained by applying a conventional lyophilisation process.

Preferred examples of polyalcohols and/or sugars according to the present invention are sucrose, inositol, sorbitol, lactose, maltose, mannitol or mixtures thereof.

In a preferred embodiment of the invention, the dispersing agents used are maltose and inositol, alone or in combination.

The use of dispersing agents according to the present invention particularly maltose and/or inositol, does not require any particular adjustments of the lyophilisation process parameters above and beyond the field of competence of a technician with normal experience in this field.

In an alternative embodiment of the present invention, the dispersing agent is added to the strain, or to at least one of the strains in the case of the use of a mixture of lactobacilli, prior to the formulation of the lyophilisation support.

In another embodiment of the present invention, the lyophilised strains are blended with the dispersing agent.

In one of the preferred embodiments of the present invention, the lyophilised product contains a mixture of lactobacilli and bifidobacteria including Streptococcus thermophilus, Bifidobacterium breve, Bidifobacterium longum, Bifidobacterium infantis, Lactobacillus acidophilus, Lactobacillus plantarum, Lactobacillus casei, Lactobacillus delbrueckeii subsp. bulgaricus, Bifidobacterium lactis, Lactobacillus pentosus, Lactobacillus paracasei, Lactobacillus helveticus, Lactobacillus brevis, and Lactobacillus salivarius subsp. salicinius in various combinations. A number of preferred embodiments are illustrated in the following examples.

As already mentioned, these strains can be lyophilised in mixtures of strains according to the present invention, or only one of the strains, preferably Streptococcus thermophilus, already added or not with a suitable lyophilisation protective agent, is lyophilised in the presence of the dispersing agents according to the invention and subsequently mixed with the other previously lyophilised strains.

The present invention applies to concentrations of lactobacilli and bifidobacteria starting from da 10⁹ CFU/g, typically from 10¹¹ CFU/g to 10¹² CFU/g. A preferred range is that from 1×10¹¹ CFU/g to 5×10¹¹ CFU/g.

Another embodiment of the invention envisages a mixture of lactobacilli as follows: (a) from approximately 5% to approximately 95% of the total weight of the combination as Lactobacillus pentosus and (b) from approximately 5% to approximately 95% of the total weight of the combination as at least one strain of Streptococcus thermophilus and/or Lactobacillus paracasei and/or Lactobacillus helveticus; and/or mutants or derivatives of (a) and/or (b).

One or more of the following strains can be added to the product, Lactobacillus acidophilus, Lactobacillus buchneri, Lactobacillus brevis, Lactobacillus casei, Lactobacillus catenaforme, Lactobacillus cellobiosus, Lactobacillus crispatus, Lactobacillus curvatus, Lactobacillus delbrueckii subsp. lactis, Lactobacillus delbrueckii subsp. bulgaricus, Lactobacillus delbrueckii subsp. delbrueckii, Lactobacillus jensenii, Lactobacillus leichmanii, Lactobacillus minutus, Lactobacillus plantarum, Lactobacillus rogosae, Lactobacillus salivarius, Lactobacillus salivarius subsp. salicinius, Lactobacillus plantarum, Lactobacillus alimentarius, Lactobacillus sanfranciscensis, Lactobacillus hilgardii, Lactobacillus fermentum, Lactobacillus fructivorans, Lactobacillus farciminis, Lactobacillus johnsonii, Lactobacillus reuteri, Lactobacillus sakei, Lactococcus lactis subsp. lactis, Lactococcus lactis subsp. cremoris, Lactococcus lactis subsp. diacetilactis, Leuconostoc spp., Weissella confusa, Bifidobacterium adolescentis, Bifidobacterium angulatum, Bifidobacterium bifidum, Bifidobacterium catenulatum, Bifidobacterium dentium, Bifidobacterium eriksonii, Bifidobacterium infantis, Bifidobacterium longum, Bifidobacterium plantarum, Bifidobacterium pseudocatenulatum, Bifidobacterium pseudolongum, Bifidobacterium lactis, Streptococcus lactis, Streptococcus raffinolactis, Streptococcus thermophilus, Acidaminococcus fermenta, Cytophaga fermentans, Rhodoferax fermentans, Cellulomonas fermentans and Zymomonas mobilis.

The lyophilised product can therefore be used for the preparation of various foods, for example, it can be a dairy product, or a nutritional product, a pharmaceutical product or a food or animal feed. In this latter case, an excipient can be present which is acceptable for human or veterinary use. The dairy products and nutritional products, the pharmaceutical compositions, the foods and animal feeds containing the lyophilised product described herein are the subject of the present invention. Examples of dairy products are cheeses and yogurt. Examples pharmaceutical compositions and nutritional products are sachets, capsules, and vials. In a preferred embodiment of the invention, the lyophilised product can be in the form of a medical food. What is meant by medical food is a food that is formulated in order to be formulated or administered by the enteral route under the supervision of a doctor and which is intended for the specific dietetic management of a disease condition, for which distinct nutritional requirements, based on recognised scientific principles, are established on the basis of a medical assessment. This definition corresponds to the definition in force in the United States for “medical food” (Orphan Drug Amendments; 21. U.S.C. § 360ee(b)(3).

Another subject of the present invention is a process for the preparation of the aforesaid product, said process including the lyophilisation of at least one bacterial strain at a concentration equal to or greater than 10⁹ CFU/g in the presence of one or more polyalcohols and/or sugars as dispersing agents and the subsequent formulation and/or packaging of the product.

In the process according to the present invention, if only a single Lactobacillus or bifidobacterium is lyophilised, this may later be mixed with other lactobacilli and/or bifidobacteria.

In another alternative embodiment, the product is prepared by means of mixing at least one strain of lyophilised lactobacillus and/or bifidobacterium at a concentration equal to or greater than 10⁹ CFU/g with one or more polyalcohols and/or sugars as dispersing agents and the subsequent formulation and/or packaging of the product.

Another subject of the present invention is the use of polyalcohols and/or sugars as dispersing agents in the preparation of lyophilised lactic acid bacteria and/or bidifidobacteria at a concentration equal to or greater than 10⁹ CFU/g.

In the preferred embodiment of the invention, the use of D-maltose and inositol affords further advantages from the nutritional point of view.

Inositol is little known and cannot be defined a vitamin, but it can be considered a vitaminic factor which is part of the B complex. The natural sources of inositol are wheat germs, beer yeast, soya lecithin and molasses. In addition, it is also contained in important amounts in bovine liver, brain, and heart as well as in beans and peanuts. In the human body it is present in the phospholipids and is capable of stimulating the endogenous production of lecithin by the body itself. Fat metabolism and the equilibrium of circulatory cholesterol are very sensitive to the action of inositol which is also capable of exerting a protective action at the level of the arterial circulation, by counteracting the hardening of fatty deposits within the arteries.

Other important activities of inositol can be summed up as a positive action in situations of mild arterial hypertension; an important action on nervous system cell metabolism; the ability to regulate anxiety and facilitate restorative sleep. Also worthy of note is its stimulatory action on the visceral musculature, and thus a positive action for those suffering from constipation.

Maltose is the main product of the hydrolysis of starch by the enzyme ptyalin (salivary amylase). The name maltose or malt sugar derives from the fact that this saccharide is formed by the enzymatic hydrolysis of the starch contained in malt. It is a reducing sugar because it has an anomeric function of the hemiacetalic type with anomeric hydroxyl in the beta position. The product presents itself in the form of needle-shaped crystals and is water-soluble. It is an easily digestible sugar and is therefore used in many baby foods.

Not least, in a preferred embodiment of the invention, maltose and/or inositol can be used as substitutes for lactose, thereby solving the problem of lactose intolerance.

The lyophilised product according to the present invention, thanks to its dispersibility characteristics, lends itself to use for the preparation of a product for an enteral diet, as well as for the preparation of compositions for topical administration or application, such as pharmaceutical or cosmetic formulations. Another advantageous application of the present invention is in the preparation of formulations for rectal use, such as, for example, in bowel enemas.

The following examples further illustrate the invention.

A) Lyophilisation of a Mixture of Lactobacilli and Bifidobacteria

### EXAMPLE 1

A mixture of lactobacilli and bifidobacteria was prepared consisting of 8 different strains: Streptococcus thermophilus, Bifidobacterium breve, Bidifobacterium longum, Bifidobacterium infantis, Lactobacillus acidophilus, Lactobacillus plantarum, Lactobacillus casei, and Lactobacillus bulgaricus.

In a 1,000 ml beaker equipped with a magnetic stirrer, 21 g of D-maltose are dissolved in 350 ml of demineralised water suitably cooled to 5° C. A perfectly clear yellowish solution is obtained after a few minutes' stirring. Keeping the solution cooled at a temperature from 2° C. to 5° C., 45 g of the lactobacilli mixture are added under stirring. The suspension is stirred for approximately 5 minutes keeping its temperature between 2° C. and 5° C. The suspension is then poured into an AISI 316 stainless steel plate precooled to −20° C. in the freezer.

The suspension tends to freeze practically instantaneously. The lyophilisation cycle is started for the purposes of obtaining an lyophilised product with powder humidity below 5% which is the condition necessary for achieving good, lasting microbiological stability of the bacterial mixture. The lyophilisation conditions are the following.

Edward Minifast 2000 Lyophilizer

| | |
|---|---|
| Condenser temperature: | −47° C. |
| Plate tempetrature: | −45° C. |
| Product temperature prior | −40° C. |
| to starting the vacuum cycle: | |

Lyophilisation heating ramp: 5° C. every 2 hours up to a product temperature of −5° C. The product is left for 12 hours at this temperature after which the temperature recovery ramp is resumed at a rate of 5° C. every 2 hours up to a product temperature of 25° C.

Product Unloading.

Similarly, a formulation was prepared using the same amount of inositol instead of D-maltose.

The two formulations were as follows:

Formula 1

| | |
|---|---|
| Lyophilised weight: | 60.2 g |
| KF (% H₂O) | 4.675% |
| Dispersibility: | optimal |
| (3 g lyophilised in 150 ml of water) | |

Formula 2

| | |
|---|---|
| Lyophilised weight | 60.9 g |
| KF (% H₂O) | 4.990% |
| Dispersibility: | optimal |
| (3 g lyophilised in 150 ml of water) | |

Similarly, two formulae were prepared with the following mixture of lactobacilli and bifidobacteria: Streptococcus thermophilus, Bifidobacterium breve, Bidifobacterium longum, Bifidobacterium infantis, Bifidobacterium lactis, Lactobacillus acidophilus, Lactobacillus pentosus, Lactobacillus paracasei, and Lactobacillus helveticus; and the following mixture: Streptococcus thermophilus, Bifidobacterium breve, Bidifobacterium longum, Bifidobacterium infantis, Bifidobacterium lactis, Lactobacillus acidophilus, Lactobacillus pentosus, Lactobacillus paracasei, Lactobacillus helveticus, Lactobacillus brevis and Lactobacillus salivarius subsp. salicinius.

### EXAMPLE 2

Lyophilisation of the lactobacilli and bifidobacteria mixture in the presence of diluents (D-maltose and inositol) and in the presence of surfactants.

In the light of the optimal results obtained with the products as in Example 1, an attempt was made to assess whether the addition of a surfactant during the lyophilisation could further improve the dispersibility of the bacteria. For this reason, additional tests were carried out using D-maltose and inositol as diluents and, in both cases, different concentrations of polysorbate 80 (Tween 80).

| | Quantities | | | | | | | |
|---|---|---|---|---|---|---|---|---|
| Composition | Formula 3 | | Formula 4 | | Formula 5 | | Formula 6 | |
| Mixture of 8 lactobacilli | 15 | g | 15 | g | 15 | g | 15 | g |
| as in Example 1 | | | | | | | | |
| D-maltose | 7.0 | g | 0 | g | 7.0 | g | 0 | g |
| Inositol | 0 | g | 7.0 | g | 0 | g | 7.0 | g |
| Polysorbate 80 | 0.02 | g | 0.02 | g | 1 | g | 1 | g |

By way of an example we give the preparation of Formula 3:

In a 500-ml beaker equipped with a magnetic stirrer, 0.02 g of polysorbate 80 are dissolved in 200 ml of demineralised water at 5° C. After 5 minutes' electromagnetic stirring, a slightly yellowish clear solution is obtained. Still under stirring and maintaining the temperature below 5° C., 7.0 g of D-maltose are dissolved. After a few minutes' stirring a perfectly clear yellowish solution is obtained.

Maintaining the solution cooled between 2 and 5° C., 15 g of the lactobacilli mixture are added. The suspension is stirred for approximately 5 minutes, maintaining its temperature between 2 and 5° C. The suspension is then poured into an AISI 316 stainless steel plate precooled to −20° C. in the freezer.

The lyophilisation was conducted as in Example 1

Results:

Formula 3

| | |
|---|---|
| Lyophilised weight | 20.2 g |
| KF (% H₂O) | 4.875% |
| Dispersibility: | optimal |
| (3 g lyophilised in 150 ml of water) | |

Formula 4

| | |
|---|---|
| Lyophilised weight | 19.75 g |
| KF (% H₂O) | 4.566% |
| Dispersibility: | optimal |
| (3 g lyophilised in 150 ml of water) | |

Formula 5

| | |
|---|---|
| Lyophilised weight | 18.7 g |
| KF (% H₂O) | 4.890% |
| Dispersibility: | optimal |
| (3 g lyophilised in 150 ml of water) | |

Formula 6

| | |
|---|---|
| Lyophilised weight | 18.9 g |
| KF (% H₂O) | 4.323% |
| Dispersibility: | optimal |
| (3 g liofilizzato in 150 ml acqua) | |

in the light of these results, it can be concluded that there is no substantial difference in the presence or absence of polysorbate 80. The dispersibility of formulae 1-6 is optimal in all cases.

Similar results were obtained with mixtures 9 and 11 of the strains as in Example 1.

B) Lyophilisation of a Single Bacterial Strain Streptococcus thermophilus) and Preparation of the Mixture with Other Bacterial Strains.

### EXAMPLE 3

Lyophilisation of the bacterium Streptococcus thermophilus in the presence of diluents (D-maltose and inositol) and subsequent blended with the other eight bacterial strains.

This embodiment of the invention reduces the production problems of the bacteria. In fact, it proves easier to lyophilise the main bacterial strain with diluents and then proceed with the mechanical blending with the other bacterial strains. For this purpose, a solution of unprotected Streptococcus thermophilus was used, containing approximately 15% dry substance, and a Streptococcus thermophilus solution to which the lyophilisation protective agent was added, containing approximately 23% dry substance.

To 67.5 g of a solution of unprotected Streptococcus thermophilus (approximately 15% dry substance) are added, in Formula 7, 5 g of inositol and, in Formula 8, 5 g of maltose: lyophilisation is then performed.

Similarly, to 67.5 g of a solution of protected Streptococcus thermophilus (approximately 23% dry substance) are added, in Formula 9, 8 g of inositol and, in Formula 10, 8 g of maltose: lyophilisation is then performed.

By way of an example we give the preparation of Formula 7.

In a 500-ml beaker equipped with a magnetic stirrer, 5.0 g of inositol are dissolved in 100 ml of demineralised water suitably cooled to 5° C. After a few minutes' stirring, a perfectly clear yellowish solution is obtained.

Maintaining the solution cooled between 2 and 5° C., 67.5 g of an aqueous solution of Streptococcus thermophilus are added under stirring. The opalescent suspension which forms is stirred for approximately 5 minutes, maintaining its temperature between 2 and 5° C. The suspension is then poured into an AISI 316 stainless steel plate precooled to −20° C. in the freezer.

The lyophilisation was conducted as in Example 1

Results:

Formula 7

| | |
|---|---|
| Weight of mixture | 14.2 g |
| KF (% H₂O) | 5.1% |
| Dispersibility: | optimal |
| (3 g lyophilised in 150 ml of water) | |

Formula 8

| | |
|---|---|
| Weight of mixture | 14.8 g |
| KF (% H₂O) | 4.6% |
| Dispersibility: | optimal |
| (3 g lyophilised in 150 ml of water) | |

Formula 9

| | |
|---|---|
| Weight of mixture | 22.1 g |
| KF (% H₂O) | 4.62% |
| Dispersibility: | optimal |
| (3 g lyophilised in 150 ml of water) | |

Formula 10

| | |
|---|---|
| Weight of mixture | 21.4 g |
| KF (% H₂O) | 4.83% |
| Dispersibility: | optimal |
| (3 g lyophilised in 150 ml of water) | |

The dispersibility results are optimal for all tests and, after thorough analysis, no substantial difference can be noted between the protected and unprotected lyophilised preparations of Streptococcus thermophilus.

The lyophilised products are then blended with the lactobacilli and bifidobacteria mixture consisting of Streptococcus thermophilus, Bifidobacterium breve, Bidifobacterium longum, Bifidobacterium infantis, Lactobacillus acidophilus, Lactobacillus plantarum, Lactobacillus casei, Lactobacillus bulgaricus, oppure da Streptococcus thermophilus, Bifidobacterium breve, Bidifobacterium longum, Bifidobacterium infantis, Bifidobacterium lactis, Lactobacillus acidophilus, Lactobacillus pentosus, Lactobacillus paracasei, and Lactobacillus helveticus, or Streptococcus thermophilus, Bifidobacterium breve, Bidifobacterium longum, Bifidobacterium infantis, Bifidobacterium lactis, Lactobacillus acidophilus, Lactobacillus pentosus, Lactobacillus paracasei, Lactobacillus helveticus, Lactobacillus brevis, and Lactobacillus salivarius subsp. salicinius, in such a way that there is 50% Streptococcus thermophilus in the finished formula. The mixtures prepared are given here below.

| | Quantities | | | | | | | |
|---|---|---|---|---|---|---|---|---|
| Composition | Formula 11 | | Formula 12 | | Formula 13 | | Formula 14 | |
| Mixture of | 5.0 | g | 5.0 | g | 5.0 | g | 5.0 | g |
| lactobacilli | | | | | | | | |
| Unprotected | 7.7 | g | 0 | g | 0 | g | 0 | g |
| lyophilised | | | | | | | | |
| Streptococcus | | | | | | | | |
| thermophilus | | | | | | | | |
| Formula 7 | | | | | | | | |
| Unprotected | 0 | g | 7.7 | g | 0 | g | 7.0 | g |
| lyophilised | | | | | | | | |
| Streptococcus | | | | | | | | |
| thermophilus | | | | | | | | |
| Formula 8 | | | | | | | | |
| Protected | 0 | g | 0.02 | g | 7.7 | g | 0 | g |
| lyophilised | | | | | | | | |
| Streptococcus | | | | | | | | |
| thermophilus | | | | | | | | |
| Formula 9 | | | | | | | | |
| Protected | 0 | g | 0 | g | 0 | g | 7.7 | g |
| lyophilised | | | | | | | | |
| Streptococcus | | | | | | | | |
| thermophilus | | | | | | | | |
| Formula 10 | | | | | | | | |

The preparation of the mixtures is done in a simple turbulence mixer for 20 minutes The mixture is rectified on an 8-mm net and placed in a sealed double PE bag. The following results are obtained.

Formula 11

| | |
|---|---|
| Weight of mixture | 12 g |
| KF (% H₂O) | 4.9% |
| Dispersibility | good |
| (3 g lyophilised in 150 ml of water) | |

Formula 12

| | |
|---|---|
| Weight of mixture | 11 g |
| KF (% H₂O) | 4.95% |
| Dispersibility | good |
| (3 g lyophilised in 150 ml of water) | |

Formula 13

| | |
|---|---|
| Weight of mixture | 11 g |
| KF (% H₂O) | 4.3% |
| Dispersibility | good |
| (3 g lyophilised in 150 ml of water) | |

Formula 14

| | |
|---|---|
| Weight of mixture | 10 g |
| KF (% H₂O) | 4.9% |
| Dispersibility | good |
| (3 g lyophilised in 150 ml of water) | |

As can be seen, the results obtained by lyophilising only one bacterial strain and adding to it the mixture of lactobacilli and bifidobacteria in a proportion of 50%, are in any case good. It can be concluded that there is no substantial difference between the results obtained with protected and unprotected Streptococcus thermophilus.

C) Dry Blending of the Mixture of Bacterial Strains with Diluents.

The simple blending of the eight strains of lactobacilli and bifidobacteria (Streptococcus thermophilus, Bifidobacterium breve, Bidifobacterium longum, Bifidobacterium infantis, Lactobacillus acidophilus, Lactobacillus plantarum, Lactobacillus casei, and Lactobacillus bulgaricus) or of the nine strains of lactobacilli and bificodacteria (Streptococcus thermophilus, Bifidobacterium breve, Bidifobacterium longum, Bifidobacterium infantis, Bifidobacterium lactis, Lactobacillus acidophilus, Lactobacillus pentosus, Lactobacillus paracasei, and Lactobacillus helveticus) or of the eleven strains of lactobacilli and bifidobacteria (Streptococcus thermophilus, Bifidobacterium breve, Bidifobacterium longum, Bifidobacterium infantis, Bifidobacterium lactis, Lactobacillus acidophilus, Lactobacillus pentosus, Lactobacillus paracasei, Lactobacillus helveticus, Lactobacillus brevis, and Lactobacillus salivarius subsp. salicinius) with diluents (inositol and D-maltose) has also been tested, obtaining optimal bacterial dispersibility results. The result is slightly inferior in terms of performance compared to the results obtained in the course of the A tests. If carried out with differing amounts of diluents, good results are obtained, directly proportional to the amount of diluent used.

The preparations which in all cases prove satisfactory are 50% mixtures of lactobacilli and diluent. The qualitative and quantitative formulations developed are given here below:

Quantities

| Composition | Formula 15 | Formula 16 |
|---|---|---|
| Mixture of lactobacilli | 20 g | 20 g |
| D-maltose | 20 g | 0 g |
| Inositol | 0 g | 20 g |

The preparation of the mixtures is done in a simple turbulence mixer for 20 minutes. The mixture is rectified on an 8-mm net and then placed in a sealed double PE bag. The following results are obtained.

Formula 15

| | |
|---|---|
| Weight of mixture | 38 g |
| KF (% H₂O) | 4.9% |
| Dispersibility | adeguate to good |
| (3 g lyophilised in 150 ml of water) | |

Formula 16

| | |
|---|---|
| Weight of mixture | 36 g |
| KF (% H₂O) | 4.8% |
| Dispersibility | adeguate to good |
| (3 g lyophilised in 150 ml of water) | |

In the light of all the tests conducted, the best results are believed to be those of formulae 1-6, and preferably those of formulae 1 and 2. The results of tests 11-14 were invariably good, whereas those of tests 15 and 16 were adequate to good.

### EXAMPLE 4

With the mixtures of eight, nine and eleven strains described above, the following formulations were prepared:

Children's Sachets Containing a Mixture of Eight/Nine Strains with a Dose of 150 bln CFU/Sachet

| | | |
|---|---|---|
| Strain mixture | 0.5 | g |
| Lactose | 0.352 | g |
| Sucrose | 1.5 | g |
| Acesulfame K and/or aspartame | 0.035 | g |
| Flavour | 0.100 | g |
| Silica | 0.013 | g |
| Total sachet weight | 2.5 | g |

Adult Sachets Containing a Mixture of Eight/Nine Strains with a Dose of 450 bln CFU/Sachet

| | | |
|---|---|---|
| Strain mixture | 1.5 | g |
| Maltose | 4.38 | g |
| Aspartame | 0.04 | g |
| Flavour | 0.07 | g |
| Silica | 0.01 | g |
| Total sachet weight | 6.0 | g |

The flavours that can be used are extremely varied. The best results have been obtained with a lemon flavour and a strawberry flavour.

1. Lyophilised product containing lactobacilli and bifidobacteria at a concentration equal to or greater than 10⁹ CFU/g, comprising a dispersing agent selected from the group consisting of polyalcohols, sugars and mixtures thereof.

2. Product according to claim 1, in which said dispersing agent is selected from the group consisting of sucrose, inositol, sorbitol, lactose, maltose, mannitol or a mixture thereof.

3. Product according to claim 2, in which said dispersing agent is D-maltose or inositol or a mixture thereof.

4. Product according to claim 1, in which at least one lyophilised lactobacillus or bifidobacterium strain is present at a concentration equal to or greater than 10⁹ CFU/g, said strain containing one or more polyalcohols and/or sugars as dispersing agents.

5. Product according to claim 1, in which said lactobacillus or bifidobacterium strain is selected from the group consisting of Streptococcus thermophilus, Bifidobacterium breve, Bidifobacterium longum, Bifidobacterium infantis, Lactobacillus acidophilus, Lactobacillus plantarum, Lactobacillus casei, and Lactobacillus bulgaricus.

6. Product according to claim 1, in which said lactobacillus or bifidobacterium strain is selected from the group consisting of Streptococcus thermophilus, Bifidobacterium breve, Bidifobacterium longum, Bifidobacterium infantis, Bifidobacterium lactis, Lactobacillus acidophilus, Lactobacillus pentosus, Lactobacillus paracasei, and Lactobacillus helveticus.

7. Product according to claim 1, in which said lactobacillus or bifidobacterium strain is selected from the group consisting of Streptococcus thermophilus, Bifidobacterium breve, Bidifobacterium longum, Bifidobacterium infantis, Bifidobacterium lactis, Lactobacillus acidophilus, Lactobacillus pentosus, Lactobacillus paracasei, Lactobacillus helveticus, Lactobacillus brevis, and Lactobacillus salivarius subsp. salicinius.

8. Product according to claim 4, in which said lyophilised strain is Streptococcus thermophilus.

9. Product according to claim 1, in which said lactobacilli and bifidobacteria are represented by the following mixture (a) from approximately 5% to approximately 95% of the total weight of the combination as Lactobacillus pentosus and (b) from approximately 5% to approximately 95% of the total weight of the combination as at least one strain of Streptococcus thermophilus and/or Lactobacillus paracasei and/or Lactobacillus helveticus; and/or mutants or derivatives of (a) and/or (b).

10. Product according to claim 1, in which said lactobacillus or bifidobacterium strain is selected from the group consisting of: Lactobacillus acidophilus, Lactobacillus buchneri, Lactobacillus brevis, Lactobacillus casei, Lactobacillus catenaforme, Lactobacillus cellobiosus, Lactobacillus crispatus, Lactobacillus curvatus, Lactobacillus delbrueckii subsp. lactis, Lactobacillus delbrueckii subsp. bulgaricus, Lactobacillus delbrueckii subsp. delbrueckii, Lactobacillus jensenii, Lactobacillus leichmanii, Lactobacillus minutus, Lactobacillus plantarum, Lactobacillus rogosae, Lactobacillus salivarius, Lactobacillus salivarius subsp. salicinius, Lactobacillus plantarum, Lactobacillus alimentarius, Lactobacillus sanfranciscensis, Lactobacillus hilgardii, Lactobacillus fermentum, Lactobacillus fructivorans, Lactobacillus farciminis, Lactobacillus johnsonii, Lactobacillus reuteri, Lactobacillus sakei, Lactococcus lactis subsp. lactis, Lactococcus lactis subsp. cremoris, Lactococcus lactis subsp. diacetilactis, Leuconostoc spp., Weissella confusa, Bifidobacterium adolescentis, Bifidobacterium angulatum, Bifidobacterium bifidum, Bifidobacterium catenulatum, Bifidobacterium dentium, Bifidobacterium eriksonii, Bifidobacterium infantis, Bifidobacterium longum, Bifidobacterium plantarum, Bifidobacterium pseudocatenulatum, Bifidobacterium pseudolongum, Bifidobacterium lactis, Streptococcus lactis, Streptococcus raffinolactis, Streptococcus thermophilus, Acidaminococcus fermenta, Cytophaga fermentans, Rhodoferax fermentans, Cellulomonas fermentans and Zymomonas mobilis.

11. Dairy product containing the product according to claim 1.

12. Nutritional product, pharmaceutical composition, food or animal feed containing the product according to claim 1.

13. Product according to claim 12, in which an acceptable excipient for human and veterinary use is present.

14. Product according to claim 12, formulated for topical or rectal use.

15. Product for topical use according to claim 14, which is a cosmetic composition or a medicament.

16. Product for rectal use according to claim 14, which is a bowel enema.

17. Product for rectal use according to claim 1, which is a medical food.

18. Process for the preparation of the product according to claim 1 that includes the lyophilization of at least one lactobacillus and/or bifidobacterium strain at a concentration equal to or greater than 10⁹ CFU/g in the presence of one or more polyalcohols and/or sugars as dispersing agents.

19. Process according to claim 18, in which said at least one lactobacillus and/or bifidobacterium strain is subsequently blended with other lactobacilli and/or bifidobacteria strains.

20. Process for the preparation of the product according to claim 1 further comprising mixing of at least one lyophilization lactobacillus and/or bifidobacterium strain at a concentration equal to or greater than 10⁹ CFU/g with one or more polyalcohols and/or sugars as dispersing agents.

21-27. (canceled)

28. A method of preparing a lyophilized bacteria, comprising:
lyophilizing a lactobacilli and/or a bifidobacteria at a concentration equal to or greater than 10⁹ CFU/g in the presence of a dispersing agent selected from the group consisting of polyalcohols, sugars and mixtures thereof
